(19) United States
(12) Patent Application Publication
(10) Pub. No.: US 2007/0135719 A1
(43) Pub. Date: Jun. 14, 2007

(54) Controlled range blood pressure cuff

(76) Inventor: John Booth, Tampa, FL (US)

Correspondence Address: ANDRUS, SCEALES, STARKE & SAWALL, LLP, 100 EAST WISCONSIN AVENUE, SUITE 1100, MILWAUKEE, WI, 53202, US

(21) Appl. No.: 11/298,732

(22) Filed: Dec. 9, 2005

(51) Int. Cl. A61B 5/02

(52) U.S. Cl. 600/490

(57) ABSTRACT

A blood pressure cuff is provided for use with a blood pressure monitoring system. The cuff includes a flexible elongated inflatable and deflatable sleeve sized to wrap around an individual's limb with the sleeve having opposite ends. A first fastener is attached on one of the sleeve ends and has at least one first fastening element. A second fastener is secured on the opposite sleeve end for attachment to the first fastener and has a plurality of spaced apart second fastening elements defining discrete closure positions. The second fastening elements are selectively receivable and separable with the at least one first fastening element to provide a desired closure of the sleeve at the discrete positions over a range of proper cuff sizing. One of the first and second fasteners is provided with a release handle enabling detachment of the first and second fasteners.

### FIELD OF THE INVENTION

This invention relates generally to patient monitoring apparatus, more particularly to blood pressure measuring and monitoring apparatus, and still more particularly to pressure cuffs used in conjunction with such blood pressure measuring and monitoring apparatus.

### BACKGROUND OF THE INVENTION

Noninvasive blood pressure (NIBP) measuring and monitoring apparatus or sphygmomanometers commonly used by clinicians consist of an inflatable, flexible sleeve, typically called a cuff, that is wrapped around a patient's arm, ordinarily above the elbow. Alternatively, the cuff may be applied around a patient's leg. A small bulb-type hand pump is connected to the sleeve by a flexible air tube to enable the cuff to be inflated sufficiently to temporarily to cut off blood flow pulsations in the arm below the cuff. When used with a manual pump, a common stethoscope is used to listen to the sound of blood flow in the patient's arm, the sound pick-up end of the stethoscope being positioned under lower regions of the cuff and over an artery in the patient's arm. Connected to the cuff, ordinarily through the hand pump, is a blood pressure monitor or indicating device, such as a mercury manometer or pressure gauge. The hand pump is used to pressurize the cuff until the flow of blood is cut off in the patient's arm; pressure is then slowly released from the cuff and the pressure at which the first sound of resumed blood flow (as determined by the stethoscope) is noted or recorded as the patient's systolic blood pressure. Pressure in the cuff is further reduced and the pressure at which the blood sounds first disappear is noted or recorded as the patient's diastolic blood pressure. Such cuffs may likewise be used in conjunction with automated blood pressure monitors that use multiple data processing techniques to process oscillometric data and create similar blood flow measurements. To trained individuals, the measured systolic and diastolic blood pressures, ordinarily expressed as the systolic pressure over the diastolic pressure, are particularly revealing of the patient's condition and may indicate the need for further treatment.

A recurring field complaint is that the NIBP measurement was inaccurate or unsuccessful. It is suspected that many of these complaints are attributable to clinicians using the wrong size cuffs. Blood pressure cuffs primarily use Velcro hook and loop elements to fasten the ends after the cuff has been wrapped around the patient's arm. To facilitate maximum adherence for the specified range of use, the cuffs typically incorporate enough material to provide 100 percent coverage or more between the hook and loop elements throughout the range. Realizing that the ends will probably stay attached without 100 percent coverage, users will occasionally place the cuff on a patient whose arm is bigger or smaller than specified. This results in a cuff that is either too tight or too loose, which affects the ability to properly couple pulsations to the blood pressure indicator.

A further problem resides in the tendency of lint to collect on the Velcro hook and loop elements which can affect the closure capability between the cuff ends during inflation of the cuff. This problem also causes inaccurate or interrupted blood pressure readings.

It is therefore desirable to provide a releasable fastening mechanism for a blood pressure cuff that does not allow clinicians to apply the cuff outside the intended range. It is further desirable to provide the cuff ends with a latching arrangement providing fixed indices that limit the wrapping of the cuff to an intended range so as to enhance the accuracy of blood pressure measurements. It is also desirable to provide a closure arrangement which more reliably retains the cuff ends together when the cuff is wrapped about an arm and inflated.

### SUMMARY OF THE INVENTION

The invention is directed towards a blood pressure cuff provided for use with a blood pressure monitoring system. The cuff includes a flexible, elongated inflatable and deflatable sleeve sized to wrap around an individual's limb, with the sleeve having opposite ends. A first fastener is attached on one of the sleeve ends and has at least one first fastening element. A second fastener is secured on the opposite sleeve end for attachment to the first fastener and has a plurality of spaced apart second fastening elements defining discrete closure positions. The second fastening elements are selectively receivable and separable with the at least one first fastening element to provide a desired closure of the sleeve ends at the discrete positions over a range of proper cuff sizing. One of the first and second fasteners are provided with a release handle enabling detachment of the first and second fasteners.

In a preferred embodiment, the first fastener includes a first planar section, an angled section and a second planar section, all of which are integrally joined together. The second fastener includes a planar section having a substantially entire bottom surface joined to the opposite sleeve end. The first fastening element is a bulbous-shaped male member extending from a bottom surface of the first fastener. The second fastening elements are upwardly facing, spaced apart female socket members formed in an upper surface of the second fastener. The socket members are deformable for frictionally receiving the bulbous-shaped male member. The bulbous-shaped male member is concealed in any of the socket members. The first and second fastening elements form a fixed indexing closure for the sleeve ends that limit the wrap of the cuff.

In another aspect of the invention, a blood pressure cuff is provided for use with a blood pressure monitoring system. The cuff comprises a flexible, elongated inflatable and deflatable sleeve sized to wrap around an individual's limb, with the sleeve having opposite ends. The sleeve is inflatable and deflatable throughout an entire length thereof. A first fastener is attached on one of the sleeve ends and has at least one first fastening element. A second fastener is secured on the opposite sleeve end for attachment to the second fastener and has a plurality of spaced apart second fastening elements defining discrete closure positions. The second fastening elements are selectively frictionally engagable and disengagable with the at least one first fastening element to provide a desired pressure fit closure of the sleeve ends at the discrete positions over a range of proper cuff sizing. Certain of the first and second fastening elements are concealed during frictional engagement thereof. One of the first and second fasteners is provided with a release handle enabling detachment of the first and second fasteners.

In yet another aspect of the invention, a blood pressure cuff is provided for use with a blood pressure monitoring system. The cuff includes a flexible, elongated inflatable and deflatable sleeve sized to wrap around an individual's limb with the sleeve having opposite ends. A first planar fastener is attached on one of the sleeve ends and has at least one male fastening element. A second planar fastener is secured on the opposite sleeve end for attachment to the first planar fastener and has a plurality of spaced apart female fastening elements defining discrete closure positions. The female fastening elements are selectively frictionally engaged and disengaged to provide a desired pressure fit closure of the sleeve ends at the discrete positions over a range of proper cuff sizing. The first planar fastener is provided with an offset release handle enabling detachment of the first and second fasteners. A bottom surface of the first fastener lies flush against an upper surface of the second fastener when the first and second fastening element are frictionally engaged.

### BRIEF DESCRIPTION OF THE DRAWINGS

The drawings illustrate the best mode presently contemplated of carrying out the invention.

In the drawings:

FIG. 1 is a diagrammatic view of a non-invasive blood pressure (NIBP) monitoring system incorporating a blood pressure cuff of the present invention;

FIG. 2 is a plan view of the blood pressure cuff of FIG. 1 illustrating a precision indexed fastening arrangement for the cuff, the ends of the cuff being uncoupled;

FIG. 3 is a view similar to FIG. 2, but showing ends of the cuff coupled together; and

FIG. 4 is a partial plan view of cuff ends attached by an alternative fastening arrangement.

### DETAILED DESCRIPTION

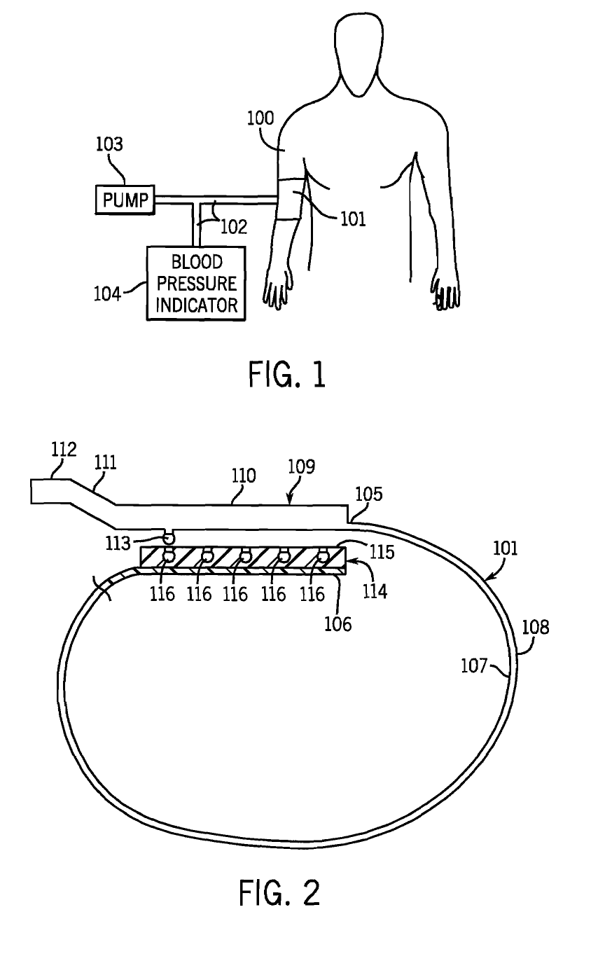

FIG. 1 illustrates a simplified version of a noninvasive blood pressure (NIBP) monitoring system. In such a system, an arm 100 of a human subject is shown wearing an inflatable and deflatable cuff 101 for occluding the brachial artery when fully inflated. The cuff 101 is connected by air tubing 102 to a pump 103 and a blood pressure indicator 104.

In a traditional monitoring system, the pump 103 is a small, bulb-type hand pump, and the indicator 104 is a mercury manometer or pressure gauge. In an automated monitoring system, the pump 103 and the blood pressure indicator 104 are formed by an arrangement of a pressure transducer, a source of pressurized air, inflate and deflate valves, a central processor and a display.

Referring to FIG. 2, the cuff 101 is formed of a flexible, elongated sleeve having opposed ends 105, 106, an inner layer 107 and an outer layer 108. The inner and outer layers 107, 108, respectively, define an inflatable and deflatable internal chamber which extends along the entire length of the sleeve from one end 105 to the opposite end 106. The chamber will allow an even application of the pressure to the entire circumference of the arm 100 for an accurate determination of blood pressure. The chamber formed by the layers 107, 108 is in communication with the air tubing 102 and the pump 103 shown in FIG. 1. It should be understood that the cuff 101 is configured with a suitable length sufficient to wrap around an arm (or leg), and a width sized to adequately cover a length of the human subject's brachial artery in the upper arm.

In accordance with the invention, the opposite ends 105, 106 of the cuff 101 are provided with cooperable and releasable fastening means designed with fixed indices that limit the wrap to the intended range of the cuff in order to enhance the accuracy of the blood pressure measurement.

Cuff end 105 is attached directly to one end of a substantially rigid first fastener 109 having a first planar section 110, an angled section 111 and a second planar section 112 integrally formed together. In the preferred embodiment, a single bulbous-shaped fastening element 113 depends from a bottom surface of the first planar section 110 at a forward end thereof.

An inflatable and deflatable length of the cuff end 106 is secured substantially along an entire bottom surface of a second generally rigid second fastener 114 having a single planar section 115. In the embodiment shown, an upper surface of the second fastener 114 is formed with a plurality of upwardly facing, spaced apart sockets that define second fastening elements 116 for selectively receiving and frictionally retaining the first fastening element 113. The sockets 116 define discrete closure positions at which the bulbous element 113 is retained. Preferably, the second fastener 114 is constructed of a deformable material which will permit the frictional engagement between first fastener 109 and second fastener 114.

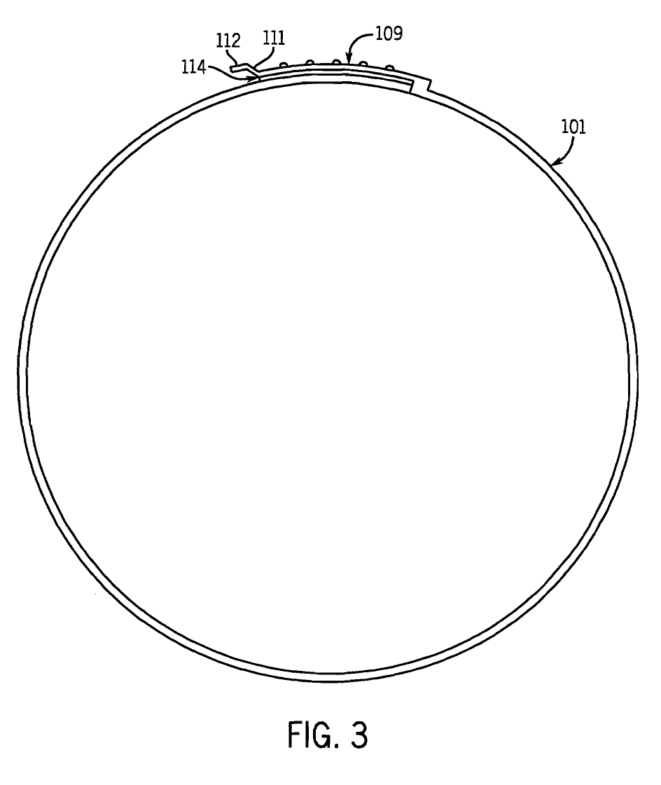

In use, a clinician wraps the cuff 101 about the arm 100 of a human subject and, as shown in FIG. 2, aligns the bulbous element 113 with a chosen one of the sockets 116 that will provide the best retention of cuff 101 over a limited range of adjustment. The clinician then applies pressure to snap or frictionally engage the male element 113 into the chosen female socket 116 as seen in FIG. 3. This motion brings the bottom surface of the first fastener 109 into contact with the upper surface of the second fastener 114. When the fastening elements 113, 116 are engaged, certain of the elements 113, 116 are concealed. The clinician then proceeds with taking the blood pressure measurement. When it is desired to uncouple the cuff ends 105, 106, the clinician conveniently grasps a release handle formed by sections 111 and 112 of the first fastener 109, and pulls the element 113 from the socket 116 so that fasteners 109 and 114 can be separated.

The invention thus provides a precision, indexing of the fastening means on the cuff ends 105, 106, and induces the clinician to focus on better fitting of the cuff on the subject's arm over an intended limited range for the particular cuff. As a result of the fastening arrangement, clinicians will be more inclined to use the proper size cuff which will increase the chances for proper, more accurate blood pressure measurement than was previously available using the prior art hook and loop cuff fasteners. Efficiency will be improved since clinicians will not be required to change cuff sizes or readjust the closure of the prior art hook and loop cuff ends and take a second blood pressure measurement. Productivity should improve when readings are successful and accurate on the first attempt.

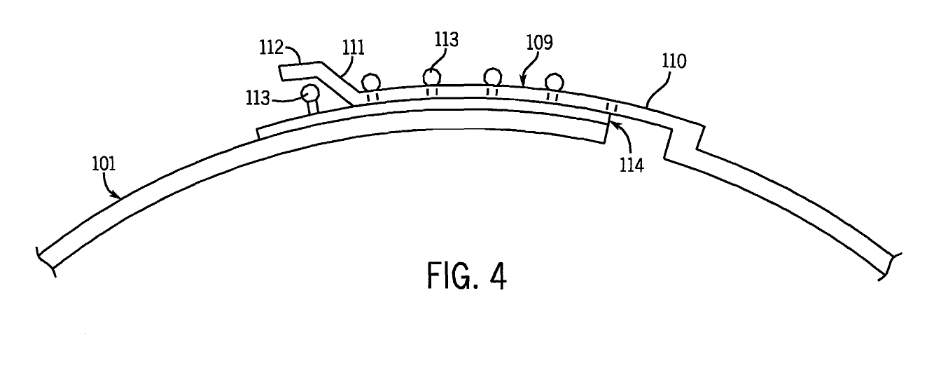

It should be fully understood, that while the preferred embodiment illustrates the first fastener 109 with a single male fastening element 113 and the second fastener 114 with a plurality of female elements 116, the invention is not limited to the particular number, shape, location or frictional engagement of the fastening elements 113, 116. For example, FIG. 4 shows a fastening arrangement in which the bulbous-shaped fastening elements 113 are located on fastener 114, and sockets 116 are formed on fastener 109. In such arrangement, the sockets 116 might be formed as indexing holes which simply receive the elements 113. Then, the protruding upper ends of the elements 113 may be engaged by a slotted strip slidably mounted along the upper surface of fastener 109 to hold the fasteners 109, 114 together. Even though the invention as shown discloses the use of a tongue and groove arrangement, it is contemplated that other similar arrangements may be utilized. One alternative example might utilize grommet holes on one of the fasteners 109, 114 and rows of cooperable releasable tabs, such as those used to adjust a ball cap, on the other of the fasteners 109, 114.

Various alternatives and embodiments are contemplated as being within the scope of the following claims particularly pointing out and distinctly claiming the subject matter regarded as the invention.

1. A blood pressure cuff for use with a blood pressure monitoring system, the cuff comprising:
a flexible elongated inflatable and deflatable sleeve sized to wrap around an individual's limb, the sleeve having opposite ends;
a first substantially rigid planar fastener externally attached on one of the sleeve ends and having at least one fastening element formed thereon; and
a second substantially rigid planar fastener externally secured on the opposite sleeve end for direct overlapping engagement with the first fastener, and having a plurality of spaced apart second fastening elements formed thereon defining discrete closure positions, the second fastening elements being selectively receivable and separable with the at least one first fastening element to provide a desired closure of the sleeve ends at the discrete positions over a range of proper cuff sizing,
wherein one of the first and second fasteners has a length that is longer than the other of the first and second fasteners and is provided with a substantially rigid release handle that extends upwardly and away from the other of the first and second fasteners enabling detachment of the first and second fasteners.

2. The blood pressure cuff of claim 1, wherein the first fastener includes a first substantially rigid planar section, a substantially rigid angled section and a second substantially rigid planar section, all of which are integrally joined together, the first planar section being formed with the at least one fastening element.

3. The blood pressure cuff of claim 1, wherein the second fastener includes a substantially rigid planar section having a substantially entire bottom surface joined externally to one planar side surface of the opposite sleeve end.

4. The blood pressure cuff of claim 1, wherein the first fastening element is a bulbous-shaped male member extending from a bottom surface of the first fastener.

5. The blood pressure cuff of claim 4, wherein the second fastening elements are upwardly facing female socket members formed in an upper surface of the second fastener.

6. The blood pressure cuff of claim 5, wherein the socket members are deformable for frictionally receiving the bulbous-shaped male member.

7. The blood pressure cuff of claim 6, wherein the bulbous-shaped male member is concealed in any of the socket members.

8. The blood pressure cuff of claim 1, wherein the first and second fastening elements form a fixed indexing closure for the sleeve ends that limit the wrap of the cuff to a proper cuff sizing.

9. The blood pressure cuff of claim 2, wherein the angled section and the second planar section define the release handle.

10. (canceled)

11. (canceled)

12. A blood pressure cuff for use with a blood pressure monitoring system, the cuff comprising:
a flexible elongated inflatable and deflatable sleeve sized to wrap around an individual's limb, the sleeve having opposite ends on inner and outer layers that define an internal chamber;
a first substantially rigid planar fastener externally attached on one of the sleeve ends and having at least one male fastening element formed thereon; and
a second substantially rigid planar fastener secured on one planar side surface of the opposite sleeve end for direct overlapping engagement with the first planar fastener and having a plurality of spaced apart female fastening elements formed thereon defining discrete closure positions, the female fastening elements being selectively frictionally engageable and disengageable with the at least one first male fastening element to provide a desired pressure fit closure of the sleeve ends at the discrete positions over a range of proper cuff sizing,
a bottom surface of the first fastener lying flush directly against an upper surface of the second fastener when the first and second fastening elements are frictionally engaged, and
wherein the first planar fastener is provided with substantially rigid offset release handle extending upwardly and away from the second planar fastener enabling detachment of the first and second fasteners.

13. (canceled)
